(19) United States
(12) Patent Application Publication
(10) Pub. No.: US 2010/0136024 A1
(43) Pub. Date: Jun. 3, 2010

(54) Selective Inhibition of Histone Deacetylase 6 for Ocular Neuroprotection or for Treatment or Control of Glaucoma

(76) Inventor: Stephen P. Bartels, Pittsford, NY (US)

Correspondence Address: Bausch & Lomb Incorporated, One Bausch & Lomb Place, Rochester, NY, 14604-2701, US

(21) Appl. No.: 12/581,340

(22) Filed: Oct. 19, 2009

Related U.S. Application Data
(60) Provisional application No. 61/114,736, filed on Nov. 14, 2008.

(51) Int. Cl. A61K 39/395; C07D 263/46; A61K 31/422; C07H 21/02; A61K 31/713; C07K 16/40; A61P 27/06; A61P 25/00

(52) U.S. Cl. 424/158.1; 548/229; 514/376; 536/24.5; 514/44 A; 530/389.1

(57) ABSTRACT

A composition comprises a material capable of selectively controlling a level or activity of HDAC6 in an ocular environment for effecting ocular neuroprotection in subjects in risk of developing or worsening an ocular neurodegenerative condition, or for treating or controlling glaucoma. Such a composition can be administered to a patient in combination with another therapy directed at providing ocular neuroprotection or treating or controlling glaucoma.

### CROSS-REFERENCE

This application claims the benefit of Provisional Patent Application No. 61/114,736 filed Nov. 14, 2008 which is incorporated by reference herein.

### BACKGROUND

The present invention relates to compositions and methods for effecting ocular neuroprotection or for treating or preventing glaucoma or progression thereof. In particular, the present invention relates to such compositions comprising selective inhibitors of histone deacetylase 6 (“HDAC6”) and such methods using such selective inhibitors.

In cells, histone deacetylases (HDACs) are often part of large multiprotein complexes that are recruited to promoter sequences through their interaction with specific DNA-binding transcription factors. Reversible protein acetylation is an important post-translational modification that regulates the function of histones and many nonhistone proteins. Acetylation of histone lysine residues in the N-terminal domain of the core histones is controlled by histone acetyltransferases and HDACs and is closely connected with gene expression and cell cycle progression. The inhibition of HDACs causes histone hyperacetylation and leads to transcriptional activation of genes such as p21^{WAF/C1P1} and Gadd 45, which are associated with growth arrest and apoptosis in tumor cells.

To date, eighteen HDAC family members have been identified and are divided into two categories, i.e., zinc-dependent enzymes (HDAC 1-11) and NAD⁺-dependent enzymes (SIRTI-7). Among these, HDAC6, a zinc-dependent HDAC isoform, is unique in that it has two HDAC domains and also a C-terminal zinc finger domain that binds ubiquitin. HDAC6 also deacetylates nonhistone proteins such as α-tubulin, HSP90, and cortactin, and is involved in microtubule stabilization, molecular chaperone activity, and cell motility. Acetylated tubulin is one of the characteristics of stabilized microtubules. Thus, HDACs participate in some important cellular processes involving gene expression, cell growth, differentiation, and proliferation.

Recent studies have revealed that HDAC6 is associated with several disease states. T. Hideshima et al., “Small-Molecule Inhibition of Proteasome and Aggresome Function Induces Synergistic Antitumor Activity in Multiple Myeloma,” PNAS, Vol. 102, No. 24, 8567 (2005), reported that the inhibition of HDAC6 causes growth inhibition in multiple myeloma cells without affecting noncancerous cells, and S. Saji et al., “Significance of HDAC6 regulation via Estrogen Signaling for Cell Motility and Prognosis in Estrogen Receptor-Positive Breast Cancer,” Oncogene, Vol. 24, 4531 (2005), reported that expression of HDAC6 is induced by estrogen stimulation of estrogen receptor α (ER-α)-positive breast cancer cells.

In addition, HDAC6 inhibition has been reported to be strongly involved in neuroprotection for Huntington's disease. Y. Itoh et al., “Design, Synthesis, Structure-Selectivity Relationship, and Effect on Cancer Cells of a Novel Series of Histone Deacetylase 6-Selective Inhibitors,” J. Med. Chem., Vol. 50, 5425 (2007), citing J. P. Dompierre et al., J. Neurosci., Vol. 27, No. 13, 3571 (2007). Itoh et al. opine that HDAC6-selective inhibitors can be therapeutic agents having few side effects. Id. at 5425. Indeed, this opinion is supported by the observations of Haggarty et al., which reported that tubacin, the first known selective inhibitor of HDAC6, does not affect the level of histone acetylation, gene-expression patterns, or cell-cycle progression. S. J. Haggarty et al., Proc. Nat'l. Acad. Sci. U.S.A., Vol. 100, 4389 (2003).

Glaucoma is a group of diseases that are characterized by the death of retinal ganglion cells (“RGCs”), specific visual field loss, and optic nerve atrophy. Glaucoma is the third leading cause of blindness worldwide. An intraocular pressure (“IOP”) that is high compared to the population mean is a risk factor for the development of glaucoma. However, many individuals with high IOP do not have glaucomatous loss of vision. Conversely, there are glaucoma patients with normal IOP. Therefore, continued efforts have been devoted to elucidate the pathogenic mechanisms of glaucomatous optic nerve degeneration.

It has been postulated that optic nerve fibers are compressed by high TOP, leading to an effective physiological axotomy and problems with axonal transport. High IOP also results in compression of blood vessels supplying the optic nerve heads (“ONHs”), leading to the progressive death of RGCs. See, e.g., M. Rudzinski and H. U. Saragovi, Curr. Med. Chem.—Central Nervous System Agents, Vol. 5, 43 (2005).

Studies suggest that the initial site of damage in eyes with ocular hypertension is the lamina cribosa. Anatomic studies in DBA/2J mice support this concept. An important aspect of this observation is that both anterograde and retrograde axonal transport are blocked. Recent studies have shown there is disruption of the transport of specific molecules, including neurotrophins and their receptors and dynein motor proteins. The local involvement of glial cells residing in the ONH and lamina cribosa may be responsible for damaging the axons as well. Investigators have known that astrocytes and microglia in the ONH of the glaucomatous eye become activated and express, for example, GFAP and vinmentin (intermediate filament proteins). The glial activation further stresses the axons and further affects axonal transport. Regardless of the exact mechanism, critical neurotropins from the brain are not supplied to the ganglion cell somas and important signals from the soma are not supplied to the axonal synapse, which negatively affects survival of RGCs. Neurotrophin deprivation can induce the c-Jun stress response, as well as GSK-3 activation and these can lead to neuronal dysfunction and ultimately to neuronal apoptosis.

In addition, there is growing evidence that other molecular mechanisms also cause direct damage to RGCs: existence of high levels of neurotoxic substances such as glutamate and nitric oxide (“NO”) and pro-inflammatory processes. Id. At low concentrations, NO plays a beneficial role in neurotransmission and vasodilation, while at higher concentrations, it is implicated in having a role in the pathogenesis of stroke, demyelination, and other neurodegenerative diseases. R.N. Saha and K. Pahan, Antioxidants & Redox Signaling, Vol. 8, No. 5 & 6, 929 (2006). NO has been recognized as a mediator and regulator of inflammatory responses. It possesses cytotoxic properties and is produced by immune cells, including macrophages, with the aim of assisting in the destruction of pathogenic microorganisms, but it can also have damaging effects on host tissues. NO can also react with molecular oxygen and superoxide anion to produce

reactive nitrogen species that can modify various cellular functions. R. Korhonen et al., Curr. Drug Target—Inflam. & Allergy, Vol. 4, 471 (2005). Furthermore, oxidative stress, occurring not only in the trabecular meshwork (“TM”) but also in retinal cells, appears to be involved in the neuronal cell death affecting the optic nerve in primary open-angle glaucoma (“POAG”). A. Izzotti et al., Mutat. Res., Vol. 612, No. 2, 105 (2006).

In addition, tumor necrosis factor-α (“TNF-α”), a proinflammatory cytokine, has recently been identified to be a mediator of RGC death. TNF-α and TNF-α receptor-1 are up-regulated in experimental rat models of glaucoma. In vitro studies have further identified that TNF-α-mediated RGC death involves the activation of both receptor-mediated caspase cascade and mitochondria-mediated caspase-dependent and caspase-independent components of cell death cascade. G. Tezel and X. Yang, Expt'l Eye Res., Vol. 81, 207 (2005). Moreover, TNF-α and its receptor were found in greater amounts in retina sections of glaucomatous eyes than in control eyes of age-matched normal donors. G. Tezel et al., Invest. Opthalmol. & Vis. Sci., Vol. 42, No. 8, 1787 (2001).

Therefore, there has been growing evidence that glaucoma may have a root cause in chronic inflammation. Failure to control the insult-induced immune response can result in autoimmune pathogenesis and likely initiates or sustains glaucomatous neurodegeneration in many patients.

A traditional therapy for glaucoma has been IOP-lowering medicaments, for example, by topical administration. However, in light of new evidence, such a course of treatment may not address the inflammatory root cause of the disease that the current body of evidence suggests.

Glucocorticoids (also referred to herein as “corticosteroids”) represent one of the most effective clinical treatment for a range of inflammatory conditions, including acute inflammation. However, steroidal drugs can have side effects that threaten the overall health of the patient. Chronic administration of glucocorticoids can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

In addition, it is known that certain glucocorticoids have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Therefore, an inflammatory root cause of glaucoma would not be treated with conventional glucocorticoids, as they would exacerbate the condition they are intended to treat.

Presently, these ocular neurodegenerative conditions are not medically reversible. There have been some achievements in slowing the progression of these blinding diseases with medicaments or surgery. However, success in treating all cases of ocular degeneration and glaucoma is still elusive. Therefore, there is a continued need to provide compounds, compositions, and methods for treating or preventing glaucoma or progression thereof. In addition, it is also very desirable to provide such compounds, compositions, and methods that at least have few or only low levels of side effects.

### SUMMARY

In general, the present invention provides compositions and methods for effecting ocular neuroprotection.

In one aspect, the present invention provides compositions and methods for controlling a progression of ocular neurodegenerative conditions.

In another aspect, the present invention provides compositions and methods for treating or controlling glaucoma.

In still another aspect, such ocular neurodegenerative conditions are selected from the group consisting of glaucoma, retinitis pigmentosa, age related macular degeneration (AMD) (including wet and dry AMD), diabetic retinopathy, optic neuritis, optic neuropathy, retinal detachment, and combinations thereof.

In another aspect, such glaucoma is selected from the group consisting of primary open-angle glaucoma, primary angle-closure glaucoma, secondary open-angle glaucoma, secondary angle-closure glaucoma, pigmentary glaucoma, neovascular glaucoma, pseudophakic glaucoma, malignant glaucoma, uveitic glaucoma, glaucoma due to peripheral anterior synechia, and combinations thereof.

In still another aspect, a composition of the present invention comprises a material capable of selectively controlling the activity of HDAC6 in an ocular environment when applied thereto.

In still another aspect, a composition of the present invention comprises a material capable of selectively controlling a level of HDAC6 in an ocular environment when applied thereto.

In yet another aspect, a composition of the present invention comprises a selective HDAC6 inhibitor in an effective amount for providing ocular neuroprotection.

In a further aspect, a composition comprises a selective HDAC6 inhibitor in an effective amount for controlling an ocular neurodegenerative condition or for treating or controlling glaucoma.

In a further aspect, a composition of the present invention also comprises an additional dissociated glucocorticoid receptor agonist (“DIGRA”), a prodrug thereof, a pharmaceutically acceptable salt thereof, or a pharmaceutically acceptable ester thereof.

In still another aspect, a composition of the present invention further comprises an additional anti-inflammatory agent selected from the group consisting of non-steroidal anti-inflammatory drugs (“NSAIDs”), peroxisome proliferator-activated receptor (“PPAR”) ligands, anti-histaminic drugs, antagonists to or inhibitors of proinflammatory cytokines (such as anti-TNF, anti-interleukin, anti-NF-KB), nitric oxide synthase inhibitors, combinations thereof, and mixtures thereof.

In a further aspect, a composition of the present invention comprises a topical formulation; injectable formulation; or implantable formulation, system, or device.

In still another aspect, a method for controlling a progression of ocular neurodegenerative conditions comprises selectively controlling activation of HDAC6, a level of HDAC6, a level of HDAC6 activity, or a combinations thereof, in an ocular environment.

In yet another aspect, a method for controlling a progression of an ocular neurodegenerative condition comprises administering to an eye of a patient in need of such controlling a composition comprising a selective HDAC6 inhibitor in an amount and at a frequency sufficient to control such progression.

In another aspect, the present invention provides a method for treating or preventing glaucoma or progression thereof. The method comprises administering a composition comprising at least a selective HDAC6 inhibitor, a prodrug thereof, a pharmaceutically acceptable salt thereof, or a pharmaceutically acceptable ester thereof into a subject in need of such treatment or prevention.

In a further aspect, such administering comprises providing such a composition in the posterior segment of the eye.

Other features and advantages of the present invention will become apparent from the following detailed description and claims.

### DETAILED DESCRIPTION

As used herein, the term “control” also includes one or more of reduction, inhibition, amelioration, alleviation, prevention, stoppage, and reversing.

As used herein, the term “prodrug” means a precursor (forerunner) of a drug. A prodrug undergoes chemical conversion by metabolic processes in the body to become an active pharmacological agent.

As discussed above, it has been postulated that optic nerve fibers are compressed by high IOP, leading to an effective physiological axotomy and problems with axonal transport. High IOP also results in compression of blood vessels supplying the ONHs leading to the progressive death of RGCs. See, e.g., M. Rudzinski and H. U. Saragovi, Curr. Med. Chem.—Central Nervous System Agents, Vol. 5, 43 (2005). Recent studies have shown there is disruption of the transport of specific molecules, including neurotrophins and their receptors and dynein motor proteins. The local involvement of glial cells residing in the ONH and lamina cribosa may be responsible for damaging the axons as well. Regardless of the exact mechanism, critical neurotropins from the brain are not supplied to the ganglion cell somas and important signals from the soma are not supplied to the axonal synapse, which negatively affects survival of RGCs.

HDAC6 inhibition has been reported to be strongly involved in neuroprotection for Huntington's disease. Y. Itoh et al., J. Med. Chem., Vol. 50, 5425 (2007), citing Dompierre et al., J. Neurosci., Vol. 27, 3571 (2007). Selective HDAC6 inhibitors have shown few side effects. For example, tubacin, a selective HDAC6 inhibitor, has fewer side effects than non-selective HDAC6 inhibitors, because it does not affect the level of histone acetylation, gene-expression patterns, or cell-cycle progression. See, e.g., S. J. Haggarty et al., Proc. Nat'l. Acad. Sci. U.S.A., Vol. 100, 4389 (2003). However, it has not been suggested, nor can it be inferred from the current knowledge, that selective HDAC6 inhibitors may be used to provide ocular neuroprotection.

Hellberg (U.S. Patent Publication No. 2007/0088045) suggests the use of non-selective HDAC inhibitors, such as suberoylanilide hydroxamic acid (“SAHA”) and trichostatin A (“TSA”) for treating degenerative conditions of the eye. It is important to note that the inhibitors disclosed in Hellberg inhibit HDACs other than HDAC6, such as HDAC1, and thus create undesirable side effects such as cytotoxicity. Thus, the use of selective inhibitors of HDAC6 to treat neurodegenerative diseases of the eye, such as glaucoma, is not disclosed or contemplated.

In general, the present invention provides compositions and methods for effecting ocular neuroprotection.

In one aspect, the present invention provides compositions and methods for controlling a progression of ocular neurodegenerative conditions.

In another aspect, the present invention provides compositions and methods for treating or controlling glaucoma.

In still another aspect, such ocular neurodegenerative conditions are selected from the group consisting of glaucoma, retinitis pigmentosa, AMD (including wet and dry AMD), diabetic retinopathy, optic neuritis, optic neuropathy, retinal detachment, and combinations thereof.

In yet another aspect, such glaucoma is selected from the group consisting of primary open-angle glaucoma, primary angle-closure glaucoma, secondary open-angle glaucoma, secondary angle-closure glaucoma, pigmentary glaucoma, neovascular glaucoma, pseudophakic glaucoma, malignant glaucoma, uveitic glaucoma, glaucoma due to peripheral anterior synechia, and combinations thereof.

In one embodiment such ocular neurodegenerative conditions comprise results of optic nerve damage due to hypertensive (high intraocular pressure (“IOP”)) or normotensive glaucoma (normal IOP).

In still another aspect, a composition of the present invention comprises a material capable of selectively controlling the activity of HDAC6 in an ocular environment when applied thereto. In one embodiment, such controlling comprises selectively reducing the level of HDAC6 deacetylation in the ocular environment when the composition is applied thereto. In another embodiment, such controlling comprises selectively inhibiting the activation of HDAC6 in the ocular environment. In still another embodiment, such controlling comprises selectively inhibiting the activity of HDAC6 in the ocular environment.

In a further aspect, a composition comprises a selective inhibitor of HDAC6 in an effective amount for controlling an ocular neurodegenerative condition or for treating or controlling glaucoma.

In yet another aspect, the selective inhibitor of HDAC6 comprises a compound or material that selectively inhibits the deacetylation activity, or the activation, of HDAC6, or reduces the expression of HDAC6 (a “selective inhibitor of HDAC6”).

In a further aspect, such an inhibitor is a selective inhibitor of HDAC6. A selective inhibitor of HDAC6 suitable for use in a composition or method of the present invention has low (alternatively, insignificant) inhibiting activity toward HDACs other than HDAC6.

The methods and compositions of this invention employ a selective inhibitor of HDAC6 activity. A selective inhibitor of HDAC6 activity is any compound, agent or material that has an inhibitory effect on the activity of HDAC6, while having a minimal inhibitory effect on other HDACs, such as HDAC1, HDAC2, HDAC3 and HDAC4. An inhibitory effect means that the amount of activity of HDAC6 that is measured in an assay in the absence of an HDAC6 inhibitor is reduced when the inhibitor is added to the assay. Assays to measure HDAC6 activity are known in the art.

Non-limiting examples of selective inhibitors of HDAC6 suitable for use in a composition or method of the present invention include inhibitors disclosed in U.S. Pat. No. 7,244,853 (1,3 dioxanes) (“the '853 Patent”); and U.S. Patent Application Publication 2006/0239909 (dioxane derivatives), which are hereby incorporated by reference their its entirety.

In some embodiments, a selective inhibitor of HDAC6 suitable for use in a composition or method of this invention comprises a 1,3 dioxane derivative having Formula I, as disclosed in U.S. Pat. No. 7,244,853,

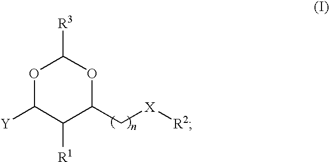

wherein
R¹ is hydrogen, or an aliphatic, alicyclic, heteroaliphatic, heterocyclic, aromatic or heteroaromatic moiety;
n is 1-5;
R² hydrogen, a protecting group, or an aliphatic, alicyclic, heteroaliphatic, heterocyclic, aromatic or heteroaromatic moiety;
X is —O—, —C(R²⁴)₂—, wherein R²⁴ is hydrogen, a protecting group, or an aliphatic, alicyclic, heteroaliphatic, heterocyclic, aromatic or heteroaromatic moiety;
or wherein two or more occurrences of R² and R²⁴, taken together, form an alicyclic or heterocyclic moiety, or an aryl or heteroaryl moiety;
R³ is an aryl or heteroaryl moiety substituted with a moiety having the structure -L-R⁴⁴, wherein L is a linker, and R⁴⁴ comprises a metal chelator;
and Y is an aromatic moiety;
and pharmaceutically acceptable salts thereof; pharmaceutically acceptable esters thereof; optical isomers thereof; mixtures of optical isomers thereof, racemic mixtures, and combinations thereof.

A subclass of the above embodiments includes compounds wherein Y is hydroxyphenyl.

An additional subclass of the above embodiments includes compounds wherein Y is hydroxyphenyl and R¹ is hydrogen.

A further subclass of the above embodiments includes compounds wherein Y is hydroxyphenyl, R¹ is hydrogen and R³ is substituted phenyl.

Such compounds can selectively inhibit HDAC6 mediated α-tubulin deacetylation. In addition, the '853 Patent provides methods of identifying which of the compounds disclosed demonstrate intercellular selectivity toward α-tubulin vs. histone deacetylation, beginning at a threshold 1.5 fold increase in acetylation levels relative to untreated cells. Id. at line 11, et seq.

Non-limiting examples of such dioxane derivatives disclosed in the '853 patent which demonstrate selective HDAC6 inhibitory activity include tubacin (also identified as JCWII114 or compound 415N3), the chemical structure of which is

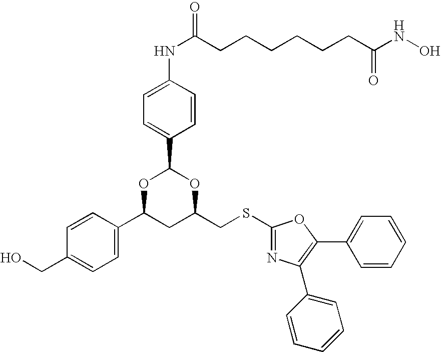

and its enantiomer JCWII169, and compound 10 of FIG. 3 thereof, below;

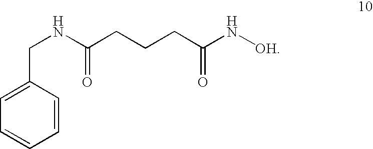

According to the '853 Patent, tubacin (compound JCWII1114) exhibited 3-fold selectivity for HDAC6 over HDAC1 and HDAC4. Id., col. 114, lines 64-65. No significant difference was noted in selectivity or potency between JCWII1114 and JCWII169. Id., col. 115, lines 37-38. Further, treatment of A549 cells with tubacin increased α-tubulin activity at concentrations as low as 125 nM, while having no effect on histone (lysine) acetylation. Id., col. 137.

The '853 Patent provides, in its FIG. 3, the following data regarding the HDAC6 selective activity of compound 10, in comparison with other preferred but non-selective inhibitors of HDAC1 and HDAC6, i.e., compounds 8 and 9:

Inhibiting Concentration IC₅₀ (μM) for

| Compound | HDAC1 | HDAC6 |
|---|---|---|
| 8 | 1.2 ± 0.5 | 0.9 ± 0.2 |
| 9 | 1.7 ± 1.2 | 1.1 ± 0.1 |
| 10 | 1.5 ± 0.5 | 0.38 ± 0.04 |

This data indicates that compound 9 is about 1.5 times, and compound 10 is about four times, more selective for HDAC6 than HDAC1.

The '853 Patent states that about 7,392 compounds were screened for selective tubulin inhibition activity (col. 115, line 48 and col. 132, line 63). An increase of tubulin bioactivity of 1.5 fold or greater was considered as the criterion for bioactivity. Two hundred seventy-three compounds were found as further non-limiting examples of such dioxane derivatives disclosed in the '853 Patent which demonstrate selective HDAC6 inhibitory activity. Id., col. 133, lines 26-29 and FIG. 29B.

Further non-limiting examples of such dioxane derivatives disclosed in the '853 patent which demonstrate selective HDAC6 inhibitory activity include 10 compounds listed as the top selective AcTubulin inhibitors of the compounds tested. '853 Patent, FIG. 29D. Those compounds are: 415N03 (tubacin), 412F01, 415E07, 415G08, 414G17, 416E17, 415I10, 418D06, 413D10 and 413F19. Their structures are:

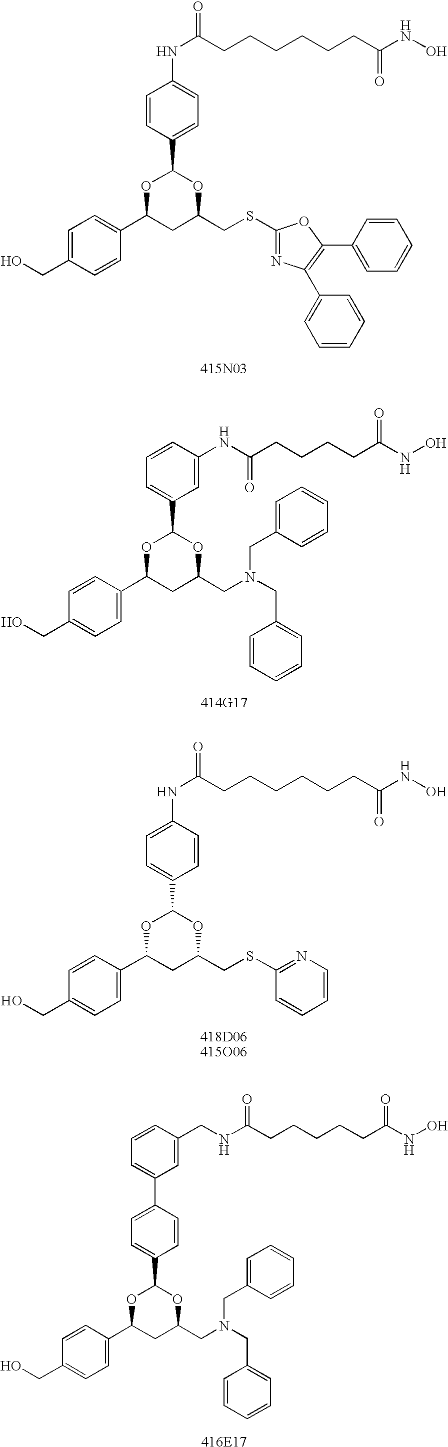

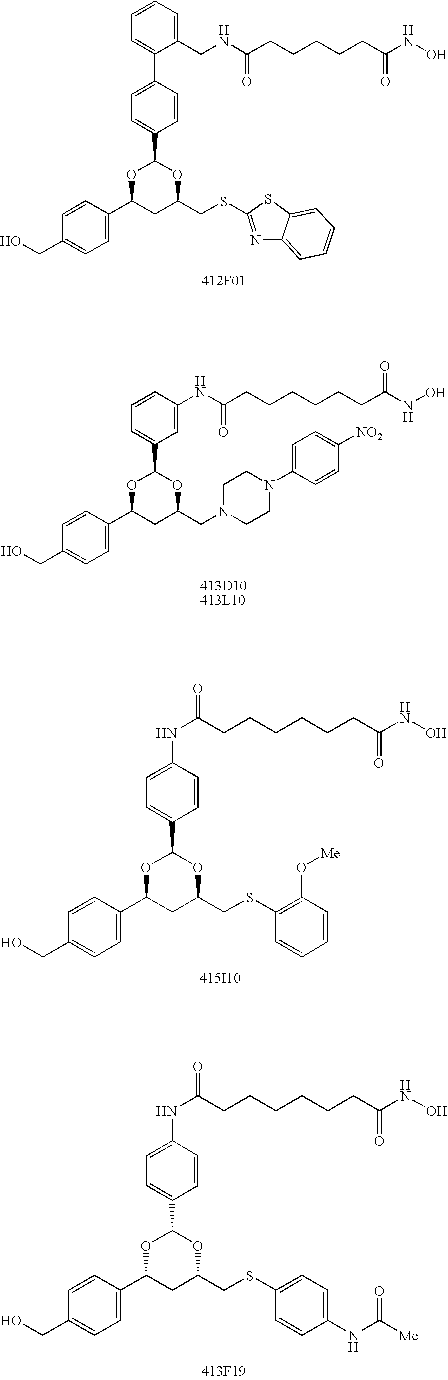

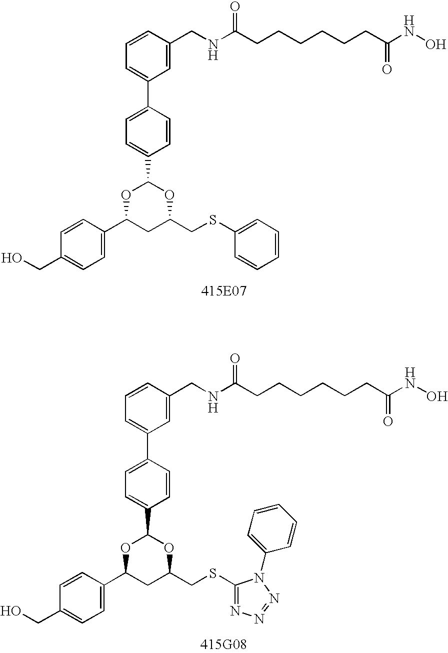

Selectivity was determined by fluorescence microscopy assay.

Other non-limiting embodiments of dioxane derivatives which demonstrate selective HDAC6 inhibitory activity suitable for use in a composition or method of the present invention are disclosed in U.S. Patent Application Publication 2006/0239909 (“the '909 Publication”), which is incorporated herein by reference and discloses a 1,3 dioxane derivative having Formula II,

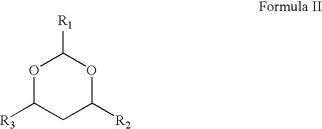

wherein
R₁ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —SR_{A}; —SOR_{A}; —SO₂R_{A}; —N(R_{A})₂; —NHC(O)R_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialklyamino, heteroarlyoxy; or heteroylthio moiety;
R₂ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —N(R_{B})₂; —NO₂; —NHC(O)R_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialklyamino, heteroarlyoxy; or heteroylthio moiety; and
R₃ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{c}; —C(═O)R_{c}; —CO₂R_{c}; —CN; —SCN; —SR_{C}; —SOR_{c}; —SO₂R_{C}; —N(R_{C})₂; —NO₂; —NHC(O)R_{C}; or —C(R_{B})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialklyamino, heteroarlyoxy; or heteroylthio moiety.

Non-limiting examples of such dioxane derivatives include:

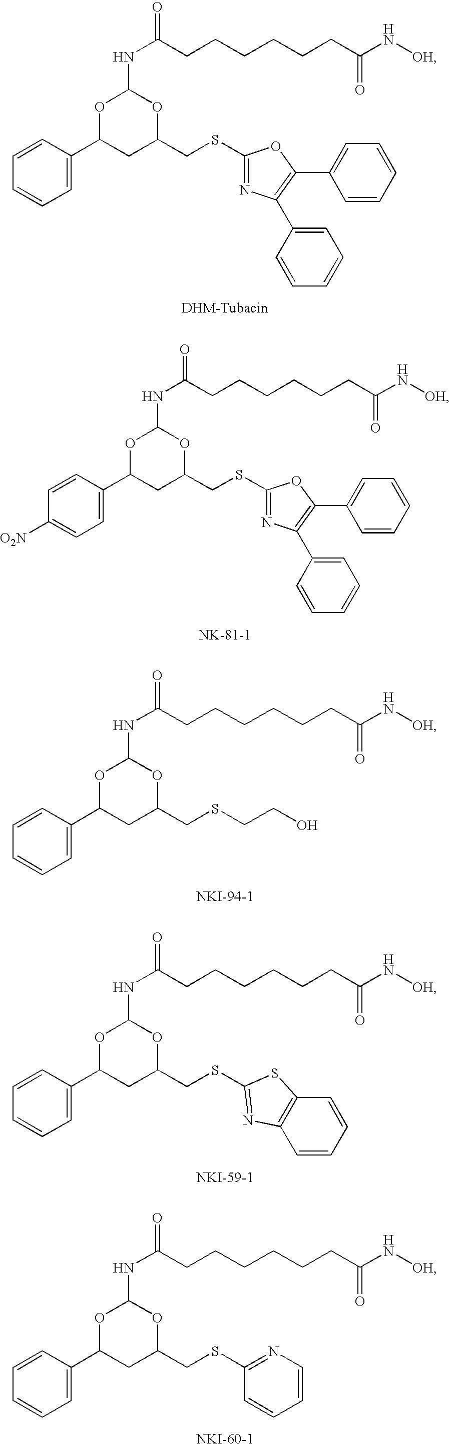

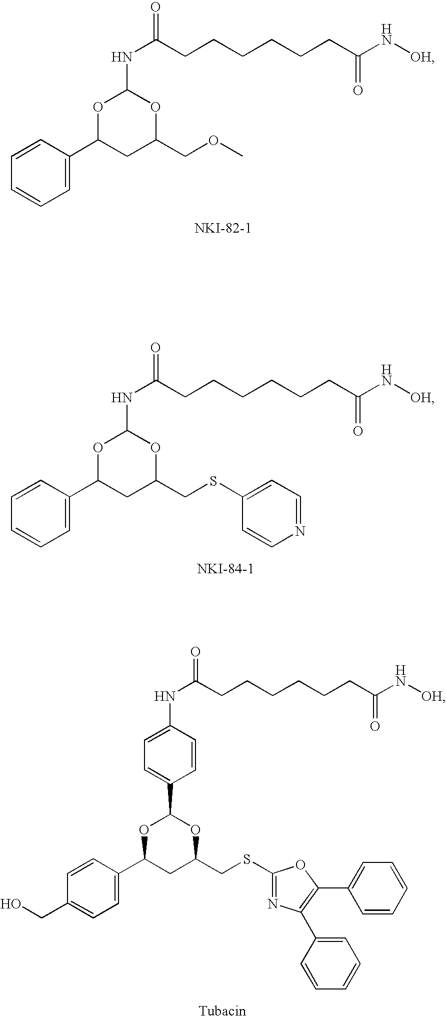

and pharmaceutically acceptable salts and derivatives thereof.

FIG. 52 of the '909 Publication provides comparative data for the non-limiting embodiments shown above. These compounds were shown to substantially inhibit the activity of HDAC6 at concentration in the range of about 1.5-25 W.

As a subclass of the above 1,3 dioxane derivatives, further non-limiting embodiments comprises a 1,3 dioxane derivative having Formula III:

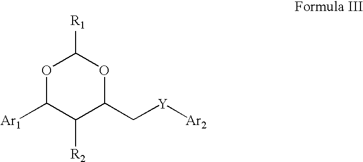

wherein

### Y is O, S, CH₂, or NR₄;

Ar₁ and Ar₂ are each independently an aryl group;
R₁ is a lower alkyl group or an aryl group;
R₂ is hydrogen, a lower alkyl group, or an aryl group; and
R₃ and R₄ are each independently hydrogen; a lower alkyl group, an aryl group, an alkylcarbonyl group, or an aminocarbonyl group.

Non-limiting examples in this subclass of such dioxane derivatives include:

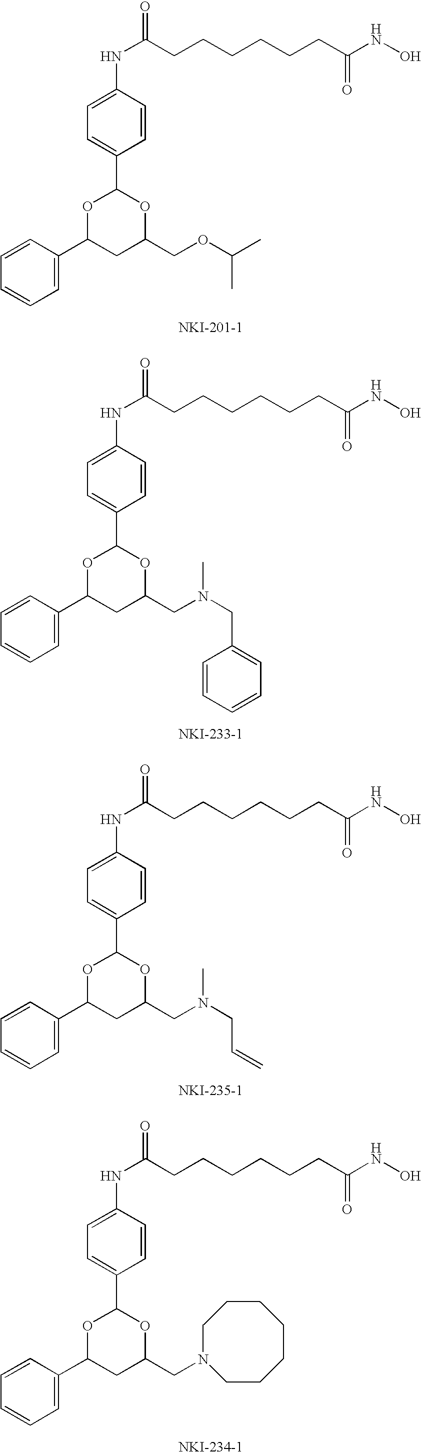

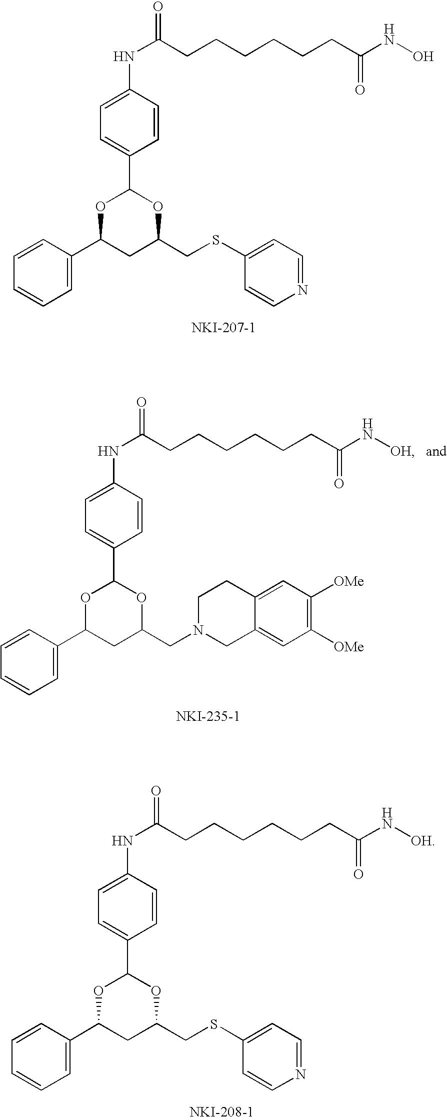

As further subclasses, non-limiting embodiments include compounds of the above Formula III, wherein: Y is S; Ar₁ is phenyl or substituted phenyl; Ar₂ is heteroaryl or oxazolyl; R₁ is phenyl or substituted phenyl (more preferably 4-aminosubstituted phenyl; or R₂ is hydrogen.

Certain compounds of the '909 Publication are selective HDAC6 inhibitors. See '909 Publication, p 29, ¶ 0243. The potency of the selective inhibitors of HDAC6 of the '909 Publication can be determined through screening assays described therein. Id., p. 38, ¶ 0331; p. 40, ¶¶ 0348-0351.

Further non-limiting examples of such selective inhibitors of HDAC6 suitable for use in a composition or method of the present invention include inhibitors disclosed in S. J. Haggarty et al., “Domain-Selective Small-Molecule Inhibitor of Histone Deacetylase 6 (HDAC6)-Mediated Tubulin Deacetylation,” Proc. Nat'l. Acad. Sci. U.S.A., Vol. 100, 4389-4394 (2003). Haggarty showed that tubacin is a selective and reversible inhibitor of α-tubulin deacetylation.

In yet other embodiments, a selective inhibitor of HDAC6 suitable for use in a composition or method of the present invention comprises substituted thiols and carbamates, as disclosed in Itoh et al., “Design, Synthesis, Structure-Selectivity Relationship, and Effect on Human Cancer Cells of a Novel Series of Histone Deacetylase 6-Selective Inhibitors,” J. Med. Chem., Vol. 50, 5425-5438 (2007), their pharmaceutically acceptable salts thereof; pharmaceutically acceptable esters thereof; optical isomers thereof; mixtures of optical isomers thereof, racemic mixtures, and combinations thereof.

Inhibitory activity of a candidate compound can be assessed by performing a Western blot assay and/or in vitro enzyme assays as disclosed in Itoh et al., supra.

Non-limiting examples of such thiol and carbamate compounds disclosed in Itoh include; (S)-S-6-(tert-butoxycarbonyl)-7-(cyclopentylamino)-7-oxoheptyl 2-methylpropanethioate, (S)-S-6-(tert-butoxycarbonyl)-7-(cyclohexylamino)-7-oxoheptyl 2-methylpropanethioate, (S)-S-6-(tert-butoxycarbonyl)-7-(cycloheptylamino)-7-oxoheptyl 2-methylpropanethioate, (S)-S-6-(tert-butoxycarbonyl)-7-(cyclobutylamino)-7-oxoheptyl 2-methylpropanethioate, (S)-S-7-(adamant-1-ylamino)-6-(tert-butoxycarbonyl)-7-(cyclopentylamino)-7-oxoheptyl 2-methylpropanethioate, (S)-tert-butyl 1-(cyclopentylamino)-7-mercapto-1-oxoheptan-2-ylcarbamate, (S)-tert-butyl 1-(cyclohexylamino)-7-mercapto-1-oxoheptan-2-ylcarbamate, (S)-tert-butyl 1-(cycloheptylamino)-7-mercapto-1-oxoheptan-2-ylcarbamate, (S)-tert-butyl 1-(tert-butylamino)-7-mercapto-1-oxoheptan-2-ylcarbamate and (S)-tert-butyl 1-(adamant-1-ylamino)-7-mercapto-1-oxoheptan-2-ylcarbamate, pharmaceutically acceptable salts thereof; pharmaceutically acceptable esters thereof; optical isomers thereof; mixtures of optical isomers thereof, racemic mixtures, and combinations thereof. See Itoh, compounds 16a-20a and 16b-20b.

To confirm HDAC6 selectivity, Itoh et al. (supra) performed in vitro enzyme assays using HDAC1, HDAC4 and HDAC6. The activity and selectivity of the compounds was compared with trichostatin A (“TSA”), also referred to in Itoh as “compound 1.”. Itoh et al. (supra) reported that HDAC6 inhibitory activity of compounds 16a-20a was similar to or greater than that of TSA “(IC₅₀ of 81 nM, 16a 29 nM, 17a 36 nM, 18a 23 nM, 19a 71 nM, 20a 82 nM).”

Further, Itoh et al. (supra) reported that “compounds 16a-20a efficiently inhibited HDAC6 in preference to HDAC1 and HDAC4 (HDAC1 IC₅₀/HDAC6 IC₅₀=35-46; HDAC4 IC₅₀/HDAC6 IC₅₀=26-51).” Moreover, the HDAC6 selectivity of these compounds was much higher than that of tubacin, which showed about a 4-fold selectivity for HDAC6 over HDAC1 and HDAC4 in enzyme assays.

The results of the enzyme assay for Itoh et al. (supra) compounds is summarized in the following table:

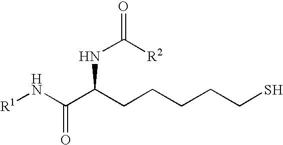

| | | | IC₅₀ (nM) | | | selectivity | |
|---|---|---|---|---|---|---|---|
| Compound | R¹ | R² | HDAC1 | HDAC4 | HDAC6 | HDAC1/HDAC6 | HDAC4/HDAC6 |
| 1 | not | not | 21 | 34 | 81 | 0.26 | 0.42 |
| | applicable | applicable | | | | | |
| 5 | not | not | no data | no data | no data | 4 | 4 |
| | applicable | applicable | | | | | |
| 7 | not | not | 48 | 32 | 41 | 1.2 | 0.78 |
| | applicable | applicable | | | | | |
| 13a | 3-biphenyl | —Ot-Bu | 62 | 38 | 54 | 1.6 | 1.0 |
| 15a | 3-quinolinyl | —Ot-Bu | 51 | 33 | 32 | 1.6 | 1.0 |
| 16a | cyclopentyl | —Ot-Bu | 1210 | 1030 | 29 | 42 | 36 |
| 17a | cyclohexyl | —Ot-Bu | 1270 | 1140 | 36 | 35 | 32 |
| 18a | cycloheptyl | —Ot-Bu | 900 | 840 | 23 | 39 | 37 |
| 19a | -t-Bu | —Ot-Bu | 3000 | 1900 | 71 | 42 | 26 |
| 20a | 1-adamantyl | —Ot-Bu | 3800 | 4200 | 82 | 46 | 51 |

In one aspect, a selective HDAC6 inhibitor included in a composition of the present invention is at least 4 times more selective for HDAC6 than for HDAC1 or HDAC4 in enzyme assays.

In another aspect, a selective HDAC6 inhibitor included in a composition of the present invention is 4-26 times more selective for HDAC6 than for HDAC1 or HDAC4 in enzyme assays.

In still another aspect, a selective HDAC6 inhibitor included in a composition of the present invention is 26-51 times more selective for HDAC6 than for HDAC1 or HDAC4 in enzyme assays.

Further, Itoh et al. (supra) prepared and compared compounds 16b-20b, in which R₁ was a bulky alkyl group, as follows:

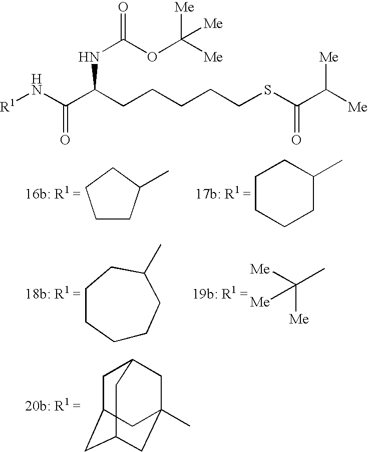

Itoh et al. (supra) concluded that “compounds 16b-20b selectively inhibit HDAC6 in preference to nuclear HDACs in cells,” based on Western blot detection of acetylated α-tubulin and acetylated histone h4 levels in HCT116 cells after 8 hours treatment. See Itoh et al., FIG. 2.

In another aspect, a selective HDAC6 inhibitor included in a composition of the present invention comprises a HDAC6 small interfering RNA (“siRNA”). Non-limiting examples of such HDAC6 siRNAs are disclosed in S. Inoue et al., “Inhibition of Histone Deacetylase Class I But Not Class II Is Critical for the Sensitization of Leukemic Cells to Tumor Necrosis Factor-Related Apoptosis-Inducing Ligand-Induced Apoptosis,” Cancer Res., Vol. 66 (13), 6785-6792 (2006) (SEQ. ID. NO. 1-6, shown below). Other non-limiting examples of HDAC6 siRNAs are disclosed in US Patent Publication No. 2007/0207950 (SEQ. ID. NO. 7, 8, shown below), which is incorporated by reference herein in its entirety. Another HDAC6 siRNA is available from Santa Cruz Biotechnology, Inc., Santa Cruz, Calif. (catalog number sc-35544).

In still another aspect, a HDAC6 inhibitor included in a composition of the present invention comprises a HDAC6 antibody raised against the full length or a fragment of human HDAC6 protein. Non-limiting examples of such a HDAC6 antibody include monoclonal or polyclonal antibodies available from Abeam Inc., Cambridge, Mass. (catalog number ab56926); Abnova Corporation, Heidelberg, Germany (catalog number H00010013-M01); Bethyl Laboratories, Montgomery, Tex. (catalog number A301-341A); and Novus Biologicals, Littleton, Colo. (catalog numbers H00010013-B01, NB100-61064, NB100-61065).

Such HDAC6 siRNAs or antibodies can be present in a composition of the present invention in the range from about 0.0001 to about 100 mg/g (or, alternatively, or from about 0.001 to about 50 mg/g, or from about 0.001 to about 25 mg/g, or from about 0.001 to about 10 mg/g, or from about 0.001 to about 5 mg/g, or from about 0.01 to about 30 mg/g, or from about 0.01 to about 25 mg/g, or from about 0.01 to about 10 mg/g, or from about 0.1 to about 10 mg/g, or from about 0.1 to about 5 mg/g).

In still another aspect, an ophthalmic pharmaceutical composition of the present invention comprises at least a selective HDAC6 inhibitor, a prodrug thereof, a pharmaceutically acceptable salt thereof, or a pharmaceutically acceptable ester thereof. Non-limiting examples of such a selective HDAC6 inhibitor include those disclosed hereinabove.

In one embodiment, the pharmaceutical composition further comprises a pharmaceutically acceptable carrier. In another aspect, said carrier is an ophthalmically acceptable carrier.

In a further aspect, a composition of the present invention comprises a topical formulation; injectable formulation; or implantable formulation, system, or device.

In another aspect, the present invention provides an ophthalmic pharmaceutical composition for effecting ocular neuroprotection, or for treating or controlling glaucoma, in a subject in need thereof. The ophthalmic pharmaceutical composition comprises at least a selective HDAC6 inhibitor, a prodrug thereof, a pharmaceutically acceptable salt thereof, or a pharmaceutically acceptable ester thereof.

In still another aspect, such ocular neuroprotection comprises controlling a progression of an ocular neurodegenerative condition.

In yet another aspect, such an ocular neurodegenerative condition is selected from the group consisting of glaucoma, retinitis pigmentosa, AMD (including wet and dry AMD), diabetic retinopathy, optic neuritis, optic neuropathy, retinal detachment, and combinations thereof.

In another aspect, such glaucoma is selected from the group consisting of primary open-angle glaucoma, primary angle-closure glaucoma, secondary open-angle glaucoma, secondary angle-closure glaucoma, pigmentary glaucoma, neovascular glaucoma, pseudophakic glaucoma, malignant glaucoma, uveitic glaucoma, glaucoma due to peripheral anterior synechia, and combinations thereof.

In one aspect, the concentration of a selective HDAC6 inhibitor, a prodrug thereof, a pharmaceutically acceptable salt thereof, or a pharmaceutically acceptable ester thereof in such an ophthalmic composition can be in the range from about 10⁻⁸ to about 500 mg/g (or, alternatively, or from about 10⁻⁷ to about 300 mg/g, or from about 10⁻⁶ to about 250 mg/g, or from about 10⁴ to about 100 mg/g, or from about 0.001 to about 50 mg/g, or from about 0.01 to about 300 mg/g, or from about 0.01 to about 250 mg/g, or from about 0.01 to about 100 mg/g, or from about 0.1 to about 100 mg/g, or from about 0.1 to about 50 mg/g).

In another aspect, the concentration of a selective HDAC6 inhibitor, a prodrug thereof, a pharmaceutically acceptable salt thereof, or a pharmaceutically acceptable ester thereof in such an ophthalmic composition can be in the range from about 10⁻⁷ to about 100 mg/g (or, alternatively, or from about 10⁻⁶ to about 50 mg/g, or from about 10⁴ to about 25 mg/g, or from about 0.001 to about 10 mg/g, or from about 0.001 to about 5 mg/g, or from about 0.01 to about 30 mg/g, or from about 0.01 to about 25 mg/g, or from about 0.01 to about 10 mg/g, or from about 0.1 to about 10 mg/g, or from about 0.1 to about 5 mg/g).

In one embodiment, an ophthalmic composition of the present invention is in a form of an emulsion, suspension, or dispersion. In another embodiment, the suspension or dispersion is based on an aqueous solution. For example, a composition of the present invention can comprise sterile saline solution. In still another embodiment, the composition comprises an oil-in-water emulsion, which can be desirable for sustained-release purposes.

In another aspect, the selective HDAC6 inhibitor, a prodrug thereof, a pharmaceutically acceptable salt thereof, or a pharmaceutically acceptable ester thereof is present in an amount effective to provide ocular neuroprotection to a subject in whom an ocular degenerative disease has begun or who has shown signs of such disease.

In still another aspect, the selective HDAC6 inhibitor, a prodrug thereof, a pharmaceutically acceptable salt thereof, or a pharmaceutically acceptable ester thereof is present in an amount effective to control a progression of an ocular neurodegenerative disease in a subject.

In yet another aspect, the selective HDAC6 inhibitor, a prodrug thereof, a pharmaceutically acceptable salt thereof, or a pharmaceutically acceptable ester thereof is present in an amount effective to control glaucoma in a subject.

In a further aspect, a composition of the present invention can further comprise an anti-inflammatory drug.

In still another aspect, such an anti-inflammatory drug can comprise a dissociated glucocorticoid receptor agonist (“DIGRA”). As used herein, a DIGRA can comprise any enantiomer of the molecule or a racemic mixture of the enantiomers.

DIGRAs can provide anti-inflammatory effects that have been seen with the well-known glucocorticoids (“GCs”), but without their accompanying side effects (such as diabetes, osteoporosis, hypertension, glaucoma, or cataract). These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol. 94, 557-572 (1998). On the other hand, there is persuasive evidence that the synthesis of IκBα, which are proteins having inhibitory effects on the NF-KB proinflammatory transcription factors, is increased by GCs. These proinflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäcke et al., Pharmacol. Ther., Vol. 96, 23-43 (2002). Thus, while the transactivation of certain genes by GCs produces beneficial effects, the transactivation of other genes by the same GCs can produce undesired side effects, one of which is glaucoma. Therefore, conventional GCs would not be employed to treat or prevent glaucoma or its progression. Consequently, it is very desirable to provide pharmaceutical compounds and compositions that produce differentiated levels of transactivation and transrepression activity on GC-responsive genes to treat or prevent glaucoma or its progression.

In still another aspect, said at least a DIGRA has Formula IV or V

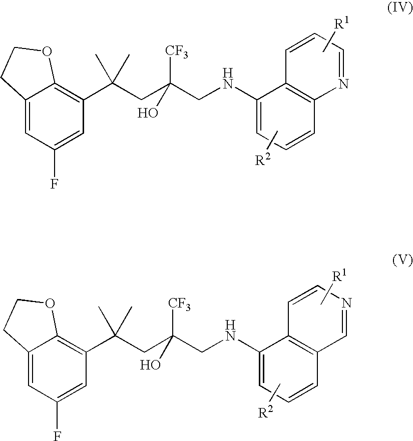

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C_{S} or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In still another embodiment, said at least a DIGRA has Formula VI.

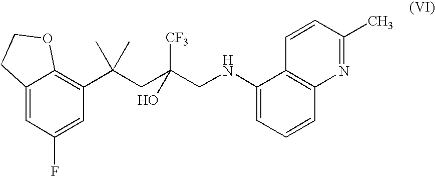

Methods for preparing compounds of Formula IV, V, or VI are disclosed, for example, in U.S. Pat. Nos. 6,897,224; 6,903,215; 6,960,581, which are incorporated herein by reference in their entirety. Still other methods for preparing such compounds also can be found in U.S. Patent Application Publication 2006/0116396, which is incorporated herein by reference, or PCT Patent Application WO 2006/050998 A1.

In another aspect, such an anti-inflammatory drug comprises a non-steroidal anti-inflammatory drug (“NSAID”). Such an anti-inflammatory drug can be present in the range from about 10⁻⁸ to about 100 mg/g (or, alternatively, or from about 0.001 to about 50 mg/g, or from about 0.001 to about 25 mg/g, or from about 0.001 to about 10 mg/g, or from about 0.001 to about 5 mg/g, or from about 0.01 to about 30 mg/g, or from about 0.01 to about 25 mg/g, or from about 0.01 to about 10 mg/g, or from about 0.1 to about 10 mg/g, or from about 0.1 to about 5 mg/g).

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), c-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

In still another embodiment, the present invention provides a method for controlling progression of optic nerve degeneration in a subject having hypertensive glaucoma. The method comprises: (a) administering a composition comprising a selective HDAC6 inhibitor, a prodrug thereof, a pharmaceutically acceptable salt thereof, or a pharmaceutically acceptable ester thereof to an eye of said subject; and (b) administering to the subject an intraocular-pressure (“IOP”) lowering drug, wherein the composition and the IOP lowering drug are administered in effective amounts at a frequency sufficient to control the progression of optic nerve degeneration. Non-limiting examples of IOP lowering drugs include prostaglandin analogs (lantanoprost, travoprost, bimatoprost), β-andrenergic receptor antagonists (timolol maleate), α₂-adrenegic agonists (brimonidine, clonidine), carbonic anhydrase inhibitors (dorzolamide, brinzolamide), cholinomimetics (pilocarpine, carbachol), and inhibitors of acetylcholinesterase such as Echothiophate (phospholine iodide).

In another aspect, an anti-inflammatory drug can comprise a peroxisome proliferator-activated receptor α (PPARα) or proliferator-activated receptor γ (PPARγ) ligand.

In another aspect, a composition of the present invention can further comprise a non-ionic or ionic surfactant. Non-limiting examples of non-ionic surfactants include polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp. 1411-1416 (Martindale, “The Complete Drug Reference,” S. C. Sweetman (Ed.), Pharmaceutical Press, London, 2005) and in Remington, “The Science and Practice of Pharmacy,” 21^{st} ed., p. 291 and the contents of chapter 22, Lippincott Williams & Wilkins, New York, 2006).

A popular group of anionic surfactants are long alkyl chain sulfonates and alkyl aryl sulfonates, such as dialkyl sodium sulfosuccinates. Alkyl sulfates are another suitable group of anionic surfactants for pharmaceutical use, such as sodium lauryl sulfate. Phospholipids comprise still another group of anionic surfactants, such as lecithin esterified to two long-chain fatty acids (often oleic, palmitic, stearic, and linoleic).

Cationic surfactants are another group that finds use in pharmaceutical formulations. Such compounds can also provide preservative effect to the formulation. Popular cationic surfactants include the quaternary ammonium compounds (such as polyquaternium-1, polyquaternium-10, benzalkonium chloride, or cetalkonium chloride) and the amine salts.

The concentration of a surfactant, when present, in a composition of the present invention can be in the range from about 0.001 to about 5 weight percent (or alternatively, from about 0.01 to about 4, or from about 0.01 to about 2, or from about 0.01 to about 1, or from about 0.01 to about 0.5, or from about 0.001 to about 0.1, or from about 0.001 to about 0.01 weight percent).

In addition, a composition of the present invention can include additives such as buffers, diluents, carriers, adjuvants, or other excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed. Water-soluble preservatives which may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5% by weight (alternatively, from about 0.01% to about 2%, or from about 0.01% to about 1% by weight). Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 2 and about 11. As such, the buffering agent may be as much as about 5% on a weight to weight basis of the total composition. Electrolytes such as, but not limited to, sodium chloride and potassium chloride may also be included in the formulation.

In one aspect, the pH of the composition is in the range from about 4 to about 9. Alternatively, the pH of the composition is in the range from about 5 to about 9, from about 6 to about 9, or from about 6.5 to about 8, or from about 5.5 to about 6.8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into an environment of an eye. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps”) or mPa·s.

In still another aspect, a material that provides an enhanced solubility of an active ingredient (a “solubility enhancer”) can be included in a composition of the present invention. Such a solubility enhancer can comprises cyclodextrin, such as α-cyclodextrin, β-cyclodextrin, γ-cyclodextrin, or a combination thereof, in anhydrous or hydrated form. Cyclodextrin derivatives are also suitable in certain embodiments, such as hydroxypropyl and sulfobutyl ether cyclodextrins, and others. Such derivatives are described for example, in U.S. Pat. Nos. 4,727,064 and 5,376,645. In addition, hydroxypropyl-β-cyclodextrin and sulfobutyl-β-cyclodextrin are commercially available. Other suitable cyclodextrin derivatives include methylated cyclodextrins, ethylated cyclodextrins, cyclodextrins with other hydroxyalkyl groups, branched cyclodextrins, cationic cyclodextrins, anionic cyclodextrins, amphoteric cyclodextrins and cyclodextrins wherein at least one glucopyranose unit has a 3,6-anhydro-cyclomalto structure.

In yet another aspect, a method for preparing a composition of the present invention comprises combining: (i) at least a selective HDAC6 inhibitor, a prodrug thereof, a pharmaceutically acceptable salt thereof, or a pharmaceutically acceptable ester thereof; and (ii) a pharmaceutically acceptable carrier. In one embodiment, such a carrier can be a sterile saline solution or a physiologically acceptable buffer. In another embodiment, such a carrier comprises a hydrophobic medium, such as a pharmaceutically acceptable oil. In still another embodiment, such as carrier comprises an emulsion of a hydrophobic material and water.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having pH in the range of, for example, from about 5.5 to about 8.5, or alternatively, from about 6.5 to about 8.0, or from about 6.5 to about 7.0, or from about 7 to about 7.5. In such embodiments, the buffer capacity of the composition desirably allows the composition to come rapidly to a physiological pH after being administered into the patient.

It should be understood that the proportions of the various components or mixtures in the following examples may be modified for the appropriate circumstances.

### Example 1

Two mixtures I and II are made separately by mixing the ingredients listed in Table 1. Five parts (by weight) of mixture I are mixed with one part (by weight) of mixture II for 15 minutes or more. The pH of the combined mixture is adjusted to 6-7.5 using 1 N NaOH or 1 N HCl to yield a composition of the present invention.

TABLE 1

| Ingredient | Amount | |
|---|---|---|
| Mixture I | | |
| Carbopol 934P NF | 0.25 | g |
| Purified water | 99.75 | g |
| Mixture II | | |
| Propylene glycol | 5 | g |
| EDTA | 0.1 | mg |
| Tubacin | 0.5 | g |

Alternatively, purified water may be substituted with an oil, such as fish-liver oil, peanut oil, sesame oil, coconut oil, sunflower oil, corn oil, or olive oil to produce an oil-based formulation comprising a selective HDAC6 inhibitor disclosed herein.

### Example 2

Two mixtures I and II are made separately by mixing the ingredients listed in Table 2. Five parts (by weight) of mixture I are mixed with two parts (by weight) of mixture II for 15 minutes or more. The pH of the combined mixture is adjusted to 6-7.5 using 1 N NaOH or 1 N HCl to yield a composition of the present invention.

TABLE 2

| Ingredient | Amount | |
|---|---|---|
| Mixture I | | |
| Diclofenac | 0.3 | g |
| Carbopol 934P NF | 0.25 | g |
| Purified water | 99.25 | g |
| Mixture II | | |
| Propylene glycol | 5 | g |
| EDTA | 0.1 | mg |
| DHM-tubacin | 0.5 | g |

### Example 3

Two mixtures I and II are made separately by mixing the ingredients listed in Table 3. Five parts (by weight) of mixture I are mixed with two parts (by weight) of mixture II for 15 minutes or more. The pH of the combined mixture is adjusted to 5.5-7.5 using 1 N NaOH or 1 N HCl to yield a composition of the present invention.

TABLE 3

| Ingredient | Amount | |
|---|---|---|
| Mixture I | | |
| Tubacin | 0.2 | g |
| Carbopol 934P NF | 0.25 | g |
| Purified water | 99.35 | g |
| Mixture II | | |
| Propylene glycol | 3 | g |
| siRNA (SEQ. ID NO. 1 & 2) | 0.25 | g |
| EDTA | 0.1 | mg |

### Example 4

Two mixtures I and II are made separately by mixing the ingredients listed in Table 4. Five parts (by weight) of mixture I are mixed with one part (by weight) of mixture II for 15 minutes or more.

TABLE 4

| Ingredient | Amount | |
|---|---|---|
| Mixture I | | |
| Tubacin | 0.3 | g |
| Carbopol 934P NF | 0.25 | g |
| Olive oil | 99.15 | g |
| Mixture II | | |
| Propylene glycol | 7 | g |
| Glycerin | 3 | g |
| NKI-59-1 | 1 | g |
| HAP (30%) | 0.5 | mg |
| PHMB | 1-20 | ppm |

Note:
“HAP” denotes hydroxyalkyl phosphates, such as those known under the trade name Dequest ®. “PHMB” denotes polyhexamethylene biguanide (a preservative).

### Example 5

The ingredients listed in Table 5 are mixed together for at least 15 minutes. The pH of the mixture is adjusted to 5.5-8 using 1 N NaOH or 1 N HCl to yield a composition of the present invention.

TABLE 5

| | Amount (% by weight, except |
|---|---|
| Ingredient | where “ppm” is indicated) |
| Povidone | 1 |
| HAP (30%) | 0.05 |
| Glycerin | 3 |
| Propylene glycol | 3 |
| (S)-tert-butyl 1-(adamant-1-ylamino)-7- | 0.5 |
| mercapto-1-oxoheptan-2-ylcarbamate | |
| Tyloxapol | 0.25 |
| BAK | 10-100 ppm |
| Purified water | q.s. to 100 |

Note:
“BAK” denotes benzalkonium chloride (a preservative).

### Example 6

The ingredients listed in Table 6 are mixed together for at least 15 minutes. The pH of the mixture is adjusted to 5.5-8 using 1 N NaOH or 1 N HCl to yield a composition of the present invention.

TABLE 6

| | Amount (% by weight, except |
|---|---|
| Ingredient | where “ppm” is indicated) |
| Povidone | 1.5 |
| HAP (30%) | 0.05 |
| Glycerin | 3 |
| Propylene glycol | 3 |
| (S)-tert-butyl 1-(cyclopentylamino)-7- | 0.75 |
| mercapto-1-oxoheptan-2-ylcarbamate | |
| Pioglitazone | 0.1 |
| Tyloxapol | 0.25 |
| PHMB | 10-50 ppm |
| Purified water | q.s. to 100 |

### Example 7

The ingredients listed in Table 7 are mixed together for at least 15 minutes.

TABLE 7

| | Amount (% by weight, except |
|---|---|
| Ingredient | where “ppm” is indicated) |
| Glycerin | 3 |
| Propylene glycol | 3 |
| NKI-84-1 | 0.25 |
| Ketorolac | 0.3 |
| PHMB | 1-5 ppm |
| Sunflower oil | q.s. to 100 |

### Example 8

The ingredients listed in Table 8 are mixed together for at least 15 minutes. The pH of the mixture is adjusted to 5.5-7.5 using 1 N NaOH or 1 N HCl to yield a composition of the present invention.

TABLE 8

| | Amount (% by weight, except |
|---|---|
| Ingredient | where “ppm” is indicated) |
| CMC (MV) | 0.5 |
| HAP (30%) | 0.05 |
| Glycerin | 3 |
| Propylene glycol | 3 |
| NKI-201-1 | 0.3 |
| Diclofenac sodium | 0.3 |
| Tyloxapol (a surfactant) | 0.25 |
| PHMB | 10-20 ppm |
| Purified water | q.s. to 100 |

### Example 9

The ingredients listed in Table 9 are mixed together for at least 15 minutes. The pH of the mixture is adjusted to 5.5-7.5 using 1 N NaOH or 1 N HCl to yield a composition of the present invention.

TABLE 9

| | Amount (% by weight, except |
|---|---|
| Ingredient | where “ppm” is indicated) |
| Glycerin | 3 |
| Propylene glycol | 3 |
| Tubacin | 0.5 |
| siRNA (SEQ. ID NO. 3 & 4) | 0.2 |
| Clofibrate (a PPARγ agonist) | 0.15 |
| PHMB | 10-50 ppm |
| Medium-chain triglyceride | 15 |
| Corn oil | q.s. to 100 |

In another aspect, one or more selective HDAC6 inhibitors, prodrugs thereof, a pharmaceutically acceptable salt thereof, or a pharmaceutically acceptable ester thereof is incorporated into a formulation for topical administration, systemic administration, periocular injection, or intravitreal injection. A formulation can desirably comprise a carrier that provides a sustained-release of the active ingredients, such as for a period longer than about 1 week (or longer than about 1, 2, 3, 4, 5, or 6 months). In certain embodiments, the sustained-release formulation desirably comprises a carrier that is insoluble or only sparingly soluble in the ocular environment. Such a carrier can be an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In one embodiment, a composition of the present invention can be injected intravitreally to control the progression of an ocular neurodegenerative disease, using a fine-gauge needle, such as 25-30 gauge. Typically, an amount from about 25 μl to about 100 μl of a composition comprising one or more selective HDAC6 inhibitors, prodrugs thereof, pharmaceutically acceptable salts thereof, or pharmaceutically acceptable esters thereof is administered into a patient. A concentration of such selective HDAC6 inhibitors, prodrugs thereof, a pharmaceutically acceptable salt thereof, or a pharmaceutically acceptable ester thereof is selected from the ranges disclosed above.

In still another aspect, one or more selective HDAC6 inhibitors, prodrugs thereof, pharmaceutically acceptable salts thereof, or pharmaceutically acceptable esters thereof is incorporated into an ophthalmic device or system that comprises a biodegradable material, and the device is implanted into the posterior cavity of a diseased eye to provide a long-term (e.g., longer than about 1 week, or longer than about 1, 2, 3, 4, 5, or 6 months) control of progression of an ocular degenerative disease. In one aspect, such control is achieved by reducing the level of HDAC6 or the level of activity of HDAC6, or by inhibiting the activation of HDAC6, in the ganglion cells over a long period of time. In certain embodiments of the present invention, the amount of one or more such HDAC6 inhibitors in such an ophthalmic device can be in the range from about 1 μg to about 10 mg, or alternatively from about 1 μg to about 5 mg.

In still another aspect, a method for controlling progression of an ocular degenerative disease comprises: (a) providing a composition comprising a selective HDAC6 inhibitor, a prodrug thereof, a pharmaceutically acceptable salt thereof, or a pharmaceutically acceptable ester thereof; and (b) administering to a subject an effective amount of the composition at a frequency sufficient to control the progression of the ocular degenerative disease.

In one embodiment, the selective HDAC6 inhibitor is selected from among those disclosed above.

In preferred embodiment, a composition of the present invention is administered intravitreally. In still another aspect, a composition of the present invention is incorporated into an ophthalmic implant system or device, and the implant system or device is surgically implanted in the vitreous cavity of the patient for the sustained or long-term release of the active ingredient or ingredients. A typical implant system or device suitable for use in a method of the present invention comprises a biodegradable matrix with the active ingredient or ingredients impregnated or dispersed therein. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918; which are incorporated herein by reference. In certain embodiments of the present invention, the amount of one or more such HDAC6 inhibitors in such an ophthalmic implant system or device can be in the range from about 1 μg to about 10 mg, or alternatively from about 1 μg to about 5 mg.

In yet another aspect, a composition of the present invention is injected into the vitreous once a month, or once every two, three, four, five, or six months. In another aspect, the composition is implanted in the patient and is replaced at a frequency of, for example, once a year or at a suitable frequency that is determined to be appropriate for controlling the progression of the ocular degenerative disease.

### Combination Therapy

A composition or a method of the present invention can be used in conjunction with other therapeutic and adjuvant or prophylactic agents commonly used to control (a) an increase of intraocular pressure, (b) a loss of neuronal cells of the retinal layers (such as retinal ganglion cells, Müller cells, amacrine cells, bipolar cells, horizontal cells, and photoreceptors) or (c) both, thus providing an enhanced overall treatment or enhancing the effects of the other therapeutic agents, prophylactic agents, and adjunctive agents used to treat and manage the different ocular neurodegenerative diseases.

High doses may be required for some currently used therapeutic agents to achieve levels to effectuate the target response, but may often be associated with a greater frequency of dose-related adverse effects. Thus, combined use of a composition of the present invention, with agents commonly used to control progression of ocular nerve damage allows the use of relatively lower doses of such other agents, resulting in a lower frequency of potential adverse side effects associated with long-term administration of such therapeutic agents. Thus, another indication of the compositions in this invention is to reduce the negative effects of protaglandins and adverse side effects of prior-art drugs used to control optic nerve degeneration, such as the development of cataracts with long-acting anticholinesterase agents including demecarium, echothiophate, and isofluorophate.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising a material capable of selectively controlling a level of HDAC6 or of HDAC6 activity in an ocular environment when applied thereto, wherein the material is present in an effective amount for effecting ocular neuroprotection.

2. The composition of claim 1, wherein the material is capable of avoiding side effects associated with non-selective inhibitors of HDACs, wherein said side effects are selected from the group consisting of affecting a level of histone acetylation, affecting gene-expression patterns, affecting cell-cycle progression, and combinations thereof.

3. The composition of claim 2, wherein said effecting neuroprotection comprises controlling progression of an ocular neurodegenerative disease.

4. The composition of claim 3, wherein said ocular neurodegenerative condition is selected from the group consisting of glaucoma, retinitis pigmentosa, wet AMD, dry AMD, diabetic retinopathy, optic neuritis, optic neuropathy, retinal detachment, and combinations thereof.

5. The composition of claim 4, wherein said glaucoma is selected from the group consisting of primary open-angle glaucoma, primary angle-closure glaucoma, secondary open-angle glaucoma, secondary angle-closure glaucoma, pigmentary glaucoma, neovascular glaucoma, normotensive glaucoma, pseudophakic glaucoma, malignant glaucoma, uveitic glaucoma, glaucoma due to peripheral anterior synechia, and combinations thereof.

6. The composition of claim 5, wherein said material comprises a selective inhibitor of HDAC6, a prodrug thereof, a pharmaceutically acceptable salt thereof, or a pharmaceutically acceptable ester thereof in combination with a second therapeutic agent, which is used for providing ocular neuroprotection or for treating or controlling glaucoma.

7. The composition of claim 5, wherein the selective inhibitor of HDAC6 displays selective inhibitory properties for HDAC6 over HDAC1 and HDAC4.

8. The composition of claim 7, wherein the selective inhibitor of HDAC6 displays selective inhibitory properties for HDAC6 over HDAC1 and HDAC4 by a factor of at least 1.5.

9. The composition of claim 7, wherein the selective inhibitor of HDAC6 displays selective inhibitory properties for HDAC6 over HDAC1 and HDAC4 by a factor of at least 3.

10. The composition of claim 7, wherein the selective inhibitor of HDAC6 displays selective inhibitory properties for HDAC6 over other HDAC1 and HDAC4 by a factor of 4 or greater.

11. The composition of claim 7, wherein the selective inhibitor of HDAC6 is selected from the group consisting of tubacin, derivatives of tubacin, 1,3 dioxane derivatives, methylpropanethioate analogues and 7-mercapto-1-oxoheptan-2-ylcarbamate analogues.

12. The composition of claim 7, wherein the selective inhibitor of HDAC6 comprises tubacin.

13. The composition of claim 7, wherein the selective inhibitor of HDAC6 comprises a HDAC6 siRNA.

14. The composition of claim 7, wherein the selective inhibitor of HDAC6 comprises a HDAC6 antibody.

15. The composition of claim 7, wherein the composition further comprises a material selected from the group consisting of NSAIDs, DIGRAs, IOP lowering drugs, and combinations thereof.

16. The composition of claim 7, wherein said composition is injectable or implantable.

17. A method for effecting ocular neuroprotection, or for treating or controlling glaucoma, in a subject in need thereof, the method comprising administering to an ocular environment of said subject a composition that comprises a selective inhibitor of HDAC6 in an effective amount and at an effective frequency to effect said neuroprotection or to treat or control said glaucoma.

18. The method of claim 17, wherein said effecting ocular neuroprotection comprises controlling a progression of an ocular neurodegenerative condition.

19. The method of claim 18, wherein said ocular neurodegenerative condition is selected from the group consisting of glaucoma, retinitis pigmentosa, wet AMD, dry AMD, diabetic retinopathy, optic neuritis, optic neuropathy, retinal detachment, and combinations thereof.

20. The method of claim 19, wherein said glaucoma is selected from the group consisting of primary open-angle glaucoma, primary angle-closure glaucoma, secondary open-angle glaucoma, secondary angle-closure glaucoma, pigmentary glaucoma, neovascular glaucoma, pseudophakic glaucoma, malignant glaucoma, uveitic glaucoma, glaucoma due to peripheral anterior synechia, and combinations thereof.

21. The method of claim 18, wherein said neurodegenerative condition results from hypertensive or normotensive glaucoma.

22. The method of claim 17, wherein the selective inhibitor of HDAC6 comprises tubacin, a prodrug, a pharmaceutically acceptable salt, or a pharmaceutically acceptable ester thereof.

23. The method of claim 17, wherein the selective inhibitor of HDAC6 comprises a HDAC6 siRNA.

24. The method of claim 17, wherein the selective inhibitor of HDAC6 comprises a HDAC6 antibody.

25. The method of claim 17, wherein said administering to an ocular environment comprises administering into a vitreous of said subject.

26. The method of claim 25, wherein said administering comprises injecting a composition or implanting an implant, device, or system, and said composition, implant, device or system comprises said selective inhibitor of HDAC6, a prodrug, a pharmaceutically acceptable salt, or a pharmaceutically acceptable ester thereof.

27. The method of claim 17, wherein said administering is performed in conjunction with another therapy or medical procedure directed to control progression of an ocular neurodegenerative condition.

28. The method of claim 17, wherein said composition further comprises a material selected from the group consisting of NSAIDs, DIGRAs, IOP-lowering drugs, and combinations thereof.


SEQUENCE LISTING (8 sequences)

SEQ ID NO: 1 - RNA, 19 nt, Artificial sequence
  Synthetic sequence, HDAC6 siRNA sense
gcucggccaagcaauggaa

SEQ ID NO: 2 - RNA, 19 nt, Artificial sequence
  Synthetic sequence, HDAC6 siRNA antisense
uuccauugcuuggccgagc

SEQ ID NO: 3 - RNA, 19 nt, Artificial sequence
  Synthetic sequence, HDAC6 siRNA sense
gcaguuaaaugaauuccau

SEQ ID NO: 4 - RNA, 19 nt, Artificial sequence
  Synthetic sequence, HDAC6 siRNA antisense
auggaauucauuuaacugc

SEQ ID NO: 5 - RNA, 19 nt, Artificial sequence
  Synthetic sequence, HDAC6 siRNA sense
gccuagaauauauugaucu

SEQ ID NO: 6 - RNA, 19 nt, Artificial sequence
  Synthetic sequence, HDAC6 siRNA antisense
agaucaauauauucuaggc

SEQ ID NO: 7 - DNA, 21 bp, Artificial sequence
  Synthetic sequence, nucleotide sequence for one siRNA strand
aatctagcggaggtaaagaag

SEQ ID NO: 8 - DNA, 21 bp, Artificial sequence
  Synthetic sequence, nucleotide sequence for one siRNA strand
aagacctaatcgtgggactgc